(19) United States
(12) Patent Application Publication
(10) Pub. No.: US 2026/0042087 A1
(43) Pub. Date: Feb. 12, 2026

(54) METAL-BASED PHOTOCATALYSIS WITH DOPED SEMICONDUCTOR SUPPORT STRUCTURES

(71) Applicant: The Regents of the University of Michigan, Ann Arbor, MI (US)

(72) Inventors: Peng Zhou, Ann Arbor, MI (US); Zetian Mi, Ann Arbor, MI (US)

(21) Appl. No.: 19/104,293

(22) PCT Filed: Aug. 17, 2023
(86) PCT No.: PCT/US2023/030470

§ 371 Date: Feb. 17, 2025

Related U.S. Application Data
(60) Provisional application No. 63/398,658, filed on Aug. 17, 2022.

(51) Int. Cl. B01J 23/72; B01J 23/50; B01J 35/33; B01J 35/39; B01J 35/45; B01J 37/02; C07C 27/12

(52) U.S. Cl. CPC B01J 23/72; B01J 23/50; B01J 35/33; B01J 35/39; B01J 35/45; B01J 37/0215; C07C 27/12

(57) ABSTRACT

A photocatalytic device includes a substrate and an array of conductive projections supported by the substrate and extending outward from the substrate, each conductive projection of the array of conductive projections having a semiconductor composition configured for photogeneration of charge carriers. Each conductive projection of the array of conductive projections is decorated with a catalyst arrangement. The catalyst arrangement includes metal nanoparticles. The semiconductor composition is doped p-type

### CROSS-REFERENCE TO RELATED APPLICATION

This application claims the benefit of U.S. provisional application entitled “Metal-Based Photocatalysis with Doped Semiconductor Support Structures,” filed Aug. 17, 2022, and assigned Ser. No. 63/398,658, the entire disclosure of which is hereby expressly incorporated by reference.

### STATEMENT REGARDING FEDERALLY SPONSORED RESEARCH OR DEVELOPMENT

This invention was made with government support under Contract No. W911NF-21-1-0337 awarded by the U.S. Army. The government has certain rights in the invention.

### BACKGROUND OF THE DISCLOSURE

### Field of the Disclosure

The disclosure relates generally to photocatalytic and other devices.

### Brief Description of Related Technology

Methane, a highly stable and inert organic fossil fuel, is widely used in the current chemical industry. However, during its chemical reforming process, the high dissociation energy (439 KJ mol⁻¹) in the cleaving step of the first C—H bond involves harsh reaction conditions, such as high reaction temperature and pressure, which undesirably increase the cost of methane reforming. The harsh reaction conditions also decrease product selectivity in connection with metastable or active liquid molecules, which often have a higher value than stable molecules produced by the process.

Photocatalysis technology, which only consumes sunlight to catalyze the chemical reaction, provides a mild approach to reform methane into high-value products. However, those high-value products, such as methanol and acetic acid, often present more active chemical structure than methane. Thus, the produced methanol is often further oxidized by active free radicals into stable products, such as CO or even CO2, which greatly decreases the value of product of the process.

Many works have been directed to improving the photocatalytic selectivity on high-value methanol. Recently, Ma et al. used hydrogen peroxide as the oxidant to reform methane into CH₃OH with a selectivity of about 90%. However, the consumption of hydrogen peroxide undesirably limited its practical industrial application. The instability of hydrogen peroxide in the photocatalytic reaction also led to a low reforming rate of 352 μmol g⁻¹ h⁻¹. Moreover, the utilization of liquid water also undesirably increased the cost of separation of water and methanol. Considering those issues, Ye et al. directly used the ubiquitous gaseous oxygen to produce —OH radicals, which could oxidize methane into methanol with a high reaction rate of 2.0 mmol g⁻¹ h⁻¹ in the photocatalytic reaction. However, poor activity was often observed in the photocatalytic methane reforming. In those examples, the overoxidation of CH₃OH by the active ·OH or ·O₂⁻radicals was considered as the reason for the low reaction rate and selectivity on the targeted products.

### SUMMARY OF THE DISCLOSURE

In accordance with one aspect of the disclosure, a photocatalytic device includes a substrate and an array of conductive projections supported by the substrate and extending outward from the substrate, each conductive projection of the array of conductive projections having a semiconductor composition configured for photogeneration of charge carriers. Each conductive projection of the array of conductive projections is decorated with a catalyst arrangement. The catalyst arrangement includes metal nanoparticles. The semiconductor composition is doped p-type.

In accordance with another aspect of the disclosure, a method of catalyzing a chemical reaction with the photocatalytic device as specified above, or as otherwise described and/or as claimed herein. The method includes irradiating the photocatalytic device with radiation, the radiation including infrared light, visible light, and ultraviolet light and, while the photocatalytic device is irradiated with the radiation, immersing the photocatalytic device in a feed gas.

In accordance with yet another aspect of the disclosure, a method of fabricating a photocatalytic device includes providing a substrate having a surface, forming an array of conductive projections on the substrate such that each conductive projection of the array of conductive projections extends outward from the substrate, each conductive projection of the array of conductive projections having a semiconductor composition configured photogeneration of charge carriers, and decorating each conductive projection of the array of conductive projections with a catalyst arrangement. Decorating each conductive projection of the array of conductive projections includes depositing metal nanoparticles on each conductive projection of the array of conductive projections. Forming the array of conductive projections includes doping the semiconductor composition p-type.

In accordance with still yet another aspect of the disclosure, a method of catalyzing a chemical reaction includes providing a catalytic device including an array of conductive projections supported by a substrate and extending outward from the substrate, each conductive projection of the array of conductive projections being decorated with a catalyst arrangement, the catalyst arrangement including metal nanoparticles, and immersing the catalytic device in water vapor and a feed gas.

In connection with any one of the aforementioned aspects, the devices and/or methods described herein may alternatively or additionally include or involve any combination of one or more of the following aspects or features. The metal nanoparticles include copper. The semiconductor composition has a bandgap such that solar radiation produces a thermal effect in the array of conductive projections. The semiconductor composition has a bandgap such that solar radiation leads to both the photogeneration of charge carriers and a thermal effect in the array of conductive projections. The semiconductor composition has a bandgap such that visible light does not contribute to the photogeneration of charge carriers. The semiconductor composition has a bandgap such that visible light produces a thermal effect in the array of conductive projections and ultraviolet light contributes to the photogeneration of charge carriers. Each conductive projection of the array of conductive projections includes a nanowire. The semiconductor composition has a wurtzite crystal structure with nitrogen-rich surfaces. The semiconductor composition includes GaN. The semiconductor composition includes a III-nitride semiconductor material doped with magnesium. The semiconductor composition is uniform. The catalyst arrangement is oxide-free. The metal nanoparticles include silver. The feed gas includes oxygen gas. The feed gas includes methane such that the chemical reaction includes reforming the methane into methanol. Immersing the photocatalytic device in the feed gas includes disposing the photocatalytic device in a flowing bed reaction system. Irradiating the photocatalytic device includes illuminating the photocatalytic device with solar radiation. Irradiating the photocatalytic device is implemented such that the photocatalytic device is heated to a temperature high enough to assist in the chemical reaction. The temperature is above about 180 degrees Celsius. Immersing the photocatalytic device in the feed gas includes immersing the photocatalytic device in water vapor. Depositing the metal nanoparticles includes implementing a photo-deposition procedure. Forming the array of conductive projections includes growing a plurality of nanowires via plasma-assisted molecular beam epitaxy (MBE) under nitrogen rich conditions. The metal nanoparticles include silver. The feed gas includes methane and oxygen such that the chemical reaction includes reforming the methane into methanol. The method further includes irradiating the catalytic device while the catalytic device is immersed in the water vapor and the feed gas. Irradiating the catalytic device includes illuminating the catalytic device with solar radiation.

### BRIEF DESCRIPTION OF THE DRAWING FIGURES

For a more complete understanding of the disclosure, reference should be made to the following detailed description and accompanying drawing figures, in which like reference numerals identify like elements in the figures.

FIG. 1 depicts (a) a 45°-tilted field emission scanning electron microscopy (FESEM) image of Cu/p-GaN nanowires of a photocatalytic device in accordance with one example, (b) X-ray diffraction (XRD) patterns of Cu/p-GaN, Cu/i-GaN and Cu/n-GaN nanowires, (c) a high-resolution high-angle annular dark-field scanning transmission electron microscopy (HAADF-STEM) image of Cu/p-GaN nanowires, and (d) high-resolution X-ray photoelectron spectroscope (XPS) spectra of Cu 2p states in Cu/p-GaN, Cu/i-GaN and Cu/n-GaN nanowires.

FIG. 2 depicts graphical plots of (a) CH₄/O₂ ratio-dependent methane oxidation on the as-prepared Cu/p-GaN photocatalyst of FIG. 1, (b) the photothermal effect on methane reforming, (c) methane oxidation activities of Cu/p-GaN, Cu/i-GaN, Cu/n-GaN and unloaded p-GaN nanowires with the CH₄/O₂ ratio of 1:2, and (d) a stability test of Cu/p-GaN in the flowing-bed photocatalytic OWS system under 300 W Xe lamp with concentrated light of 5,000 mW cm⁻² on a 0.64 cm² photocatalyst wafer sample. One short-pass 400 nm filter was equipped in the Xe lamp to produce the light with wavelength of <400 nm, while the light with wavelength of >400 nm was achieved by a long-pass 400 nm filter.

FIG. 3 depicts graphical plots of the in-situ IR spectrum of photocatalytic methane oxidation on (a) Cu/p-GaN, (b) Cu/i-GaN and (c) Cu/n-GaN nanowires. The CH₄/O₂ ratio of 1:2 was used in the in-situ IR test. The temperature was set to 212° C. which was consistent with the above photocatalytic methane oxidation. FIG. 3 also schematically depicts the charge density difference mappings (front view) between Cu nanoparticles and GaN surfaces in (d) Cu/p-GaN, (e) Cu/i-GaN and (f) Cu/n-GaN. The isosurface of charge density is 0.004 e Å⁻³. The yellow and sky-blue regions stand for the positive and negative charges, respectively. FIG. 3 also depicts graphical plots of (g) PDOS of Cu 3d states in Cu/p-GaN, Cu/i-GaN and Cu/n-GaN, and (h) energy profiles of methane oxidation on Cu/p-GaN, Cu/i-GaN and Cu/n-GaN. The dashed line indicates the Fermi level.

FIG. 4 is a schematic view of the mechanism of photocatalytic methane reforming into methanol with oxygen as oxidant on CuNP-loaded GaN.

FIG. 5 is a schematic view of a photocatalytic device having a catalyst support arrangement for photothermal catalytic reforming of methane into methanol in accordance with one example.

FIG. 6 is a flow diagram of a method of fabricating a photocatalytic device having a catalyst support arrangement for photothermal catalytic reforming of methane into methanol in accordance with one example.

FIG. 7 depicts (a) a 45°-tilted FESEM image, (b) an XRD pattern, (c) a HAADF-STEM image, (d) a bright-field transmission electron microscopy (TEM) image, (e) energy dispersive X-ray (EDS) elemental mapping, and (f) room-temperature PL spectrum of Ag/InGaN nanowires of a photocatalytic device in accordance with one example

FIG. 8 depicts graphical plots of (a) CH₄/O₂ ratio-dependent methane oxidation on an as-prepared Ag/InGaN photocatalyst device in the presence of water in accordance with one example, (b) methane oxidation activities of an example Ag/InGaN photocatalyst device without or with H₂O (or D₂O), (c) a stability test of an example Ag/InGaN photocatalyst device under 300 W Xe lamp with concentrated light of 5,000 mW cm⁻² on a 0.64 cm² photocatalyst wafer.

FIG. 9 depicts graphical plots of (a, b) in-situ IR spectrum of photocatalytic methane oxidation on Ag/InGaN photocatalytic devices (a) without or (b) with water, (c) high-resolution XPS spectra of Ag/InGaN before and after reaction (with circles representative of the fitting curve), (d) a free energy profile of methane oxidation on Ag/InGaN photocatalytic devices without or with water, (e) PDOS plots of Ag 4d states without or with water, and (f) PDOS plots of In 5s and 5p states without or with water (with a vertical dashed line representative of Fermi level), as well as (g, h) charge density difference mappings between methanol and Ag/InGaN surfaces (g) without or (h) with water.

The embodiments of the disclosed devices and methods may assume various forms. Specific embodiments are illustrated in the drawing and hereafter described with the understanding that the disclosure is intended to be illustrative. The disclosure is not intended to limit the invention to the specific embodiments described and illustrated herein.

### DETAILED DESCRIPTION OF THE DISCLOSURE

Photocatalytic and other catalytic devices with a catalyst support arrangement for photothermal and other catalytic methane reforming and photothermal and other catalysis of other chemical reactions are described. The catalyst support arrangement includes an array of conductive projections, such as nanowires or other nanostructures. The nanowires provide support for a metal nanoparticle-based catalyst arrangement. As described below, each nanowire has a semiconductor composition that is doped p-type. The p-type doping enhances the interaction between the metal nanoparticles and the semiconductor composition of the nanowires. In some cases, the semiconductor composition is or includes a III-nitride semiconductor material. Methods of using and fabricating such photocatalytic devices are also described.

The conductive projections of the disclosed photocatalytic devices may be configured to promote the chemical reaction thermally and via photoexcitation. The semiconductor composition may have a bandgap such that irradiation of the photocatalytic devices via, for instance, solar radiation, produces both a photothermal effect and photogeneration of charge carriers. The photothermal effect raises the operating temperature of the photocatalytic device high enough to assist the chemical reaction. The high operating temperature is useful in connection with reforming methane into liquid fuels, such as methanol. As described herein, the disclosed devices optimize the reaction pathway of CH₃OH formation, thereby improving the efficiency of photocatalytic methane reforming into methanol.

Although described in connection with methane reforming, the disclosed photocatalytic devices and corresponding methods may be used in other chemical reaction contexts. The photocatalytic devices and corresponding methods may be applied to a wide variety of chemical reactions. For instance, the disclosed photocatalytic devices may be used in connection with CO₂ reduction and N₂ fixation.

Although described in connection with solar radiation, the disclosed photocatalytic devices are useful in connection with a variety of different light sources. The spectrum or other characteristics of the light source may vary accordingly. For instance, the radiation may be or otherwise include various types of artificial light. The artificial light include any combination of infrared, visible, and/or ultraviolet wavelengths.

Although described in connection with a deployment in a gas-phase system, the disclosed photocatalytic devices are also compatible with liquid-phase systems. For instance, the photothermal effects described herein are also useful in water-phase photocatalytic water-splitting, which, in turn, may be used in connection with CO2 reduction.

Although described in connection with nanowires having a GaN semiconductor composition, the disclosed photocatalytic devices are not limited to III-nitride semiconductor materials or a uniform semiconductor compositions. For instance, other semiconductor materials, such as TiO₂ and CdS, may be used. Also for instance, the conductive projections of the photocatalytic devices may have a multi-band configuration. For example, the arrays may include monolithically integrated multiple-band InGaN nanostructures or segments configured to act as photocatalysts. Each conductive projection may thus be capable of photoexcitation via a wider range of wavelengths, including, for instance, both ultraviolet and visible portions of the solar spectra. Any number or type of segments may be included.

Although described in connection with copper nanoparticles, the enhanced metal-support interaction of the disclosed photocatalytic devices may be applied to other metal-loaded semiconductor photocatalysts. For instance, additional and/or other metal catalysts, such as silver and gold nanoparticles, may be used for methane reforming into methanol and/or other chemical reactions.

Examples of the disclosed photocatalytic devices exhibit enhanced metal-support interaction between p-type GaN support structures and Cu nanoparticles (CuNP). For instance, the enhanced interaction between a metal catalyst and the support structure may promote photothermal catalytic methane reforming into methanol in a flowing-bed system using methane and oxygen as feed gas. In-situ IR measurements and density functional theory (DFT) simulations revealed that this enhanced metal-support interaction in a CuNP-loaded p-type GaN (Cu/p-GaN) arrangement not only promotes charge separation/transfer between the CuNP catalysts and p-GaN photocatalyst, but also significantly decreases the reaction energy of rate-determining methanol desorption on catalyst surface. Thus, the enhanced metal-support interaction of the Cu/p-GaN arrangement provided a 3.5-fold higher activity for methanol production in photothermal catalytic methane reforming relative to other arrangements, i.e., CuNP-loaded intrinsic GaN (Cu/i-GaN) and n-type GaN (Cu/n-GaN).

GaN is a semiconductor photocatalyst with useful catalytic redox ability. The well-controlled growth of GaN nanostructures by plasma-assisted molecular beam epitaxy (PAMBE) provides an effective strategy for tuning the catalytic property of GaN at the electronic and atomic level.

To examine the enhanced metal-support interaction of the disclosed devices, nanowires composed of p-GaN, i-GaN and n-GaN were grown on silicon wafers using PAMBE-based procedures. Further details regarding examples of the growth procedures are set forth in Li, L. et al., “Photoinduced Conversion of Methane into Benzene over GaN,” Nanowires. J. Am. Chem. Soc., 136, 7793-7796 (2014), Kibria, M. G. et al., “Tuning the surface Fermi level on p-type gallium nitride nanowires for efficient overall water splitting,” Nat. Commun., 5, 3825 (2014), the entire disclosures of which are incorporated by reference. Room temperature photoluminescence (PL) spectra showed that all as-prepared samples shared an emission peak at about 365 nm, which is attributed to the typical band gap (3.40 eV) of wurtzite GaN.

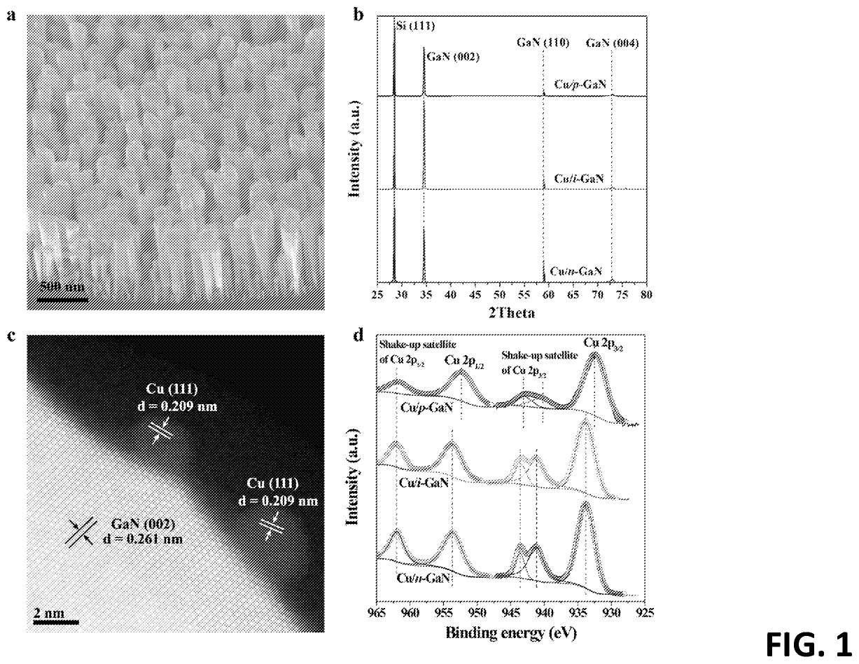

Copper nanoparticles (CuNPs) were photo-deposited on the surfaces of the p-GaN, i-GaN and n-GaN nanowires. The resulting arrangements are thus denoted as Cu/p-GaN, Cu/i-GaN and Cu/n-GaN, respectively. Field emission scanning electron microscopy (FESEM) images indicated that the well-arrayed GaN nanowires in Cu/p-GaN, Cu/i-GaN and Cu/n-GaN share an approximate length of about 700 nm on the silicon wafer (FIG. 1, part a). X-ray diffraction (XRD) patterns also confirmed that the crystal structures of all three

arrangements are wurtzite GaN nanowires grown along the direction on silicon (111) surface corresponding to the standard PDF card (2-1078), which suggests the high crystallinity and uniformity of GaN nanowires (FIG. 1, part b). It should be noted that the XRD peak of Cu nanoparticles was not observed due to its low content. According to the measurements by an inductively coupled plasma-atomic emission spectrometer (ICP-AES), the contents of the Cu nanoparticles on Cu/p-GaN, Cu/i-GaN and Cu/n-GaN arrangements were determined to be 46.9, 32.5 and 43.2 μg cm⁻², respectively. This was further confirmed by high-angle annular dark-field scanning transmission electron microscopy (HAADF-STEM) images in which a clear lattice fringe with a width of 0.261 nm was observed perpendicular to the growth direction of GaN nanowires (FIG. 1, part c). On the surface of the GaN nanowires, the metallic Cu nanoparticles exhibited a lattice distance of 0.209 nm in each of the arrangements. The Cu nanoparticles act as a catalyst (or cocatalyst) in the photocatalysis. The X-ray photoelectron spectroscope (XPS) measurements showed that the 2/3/2 state of the CuNPs on p-GaN is located in a more negative region than those on i-GaN and n-GaN (FIG. 1, part d), indicating more electrons on p-GaN-supported CuNPs. This implies an enhanced metal-support interaction between metallic CuNP and the p-GaN semiconductor composition.

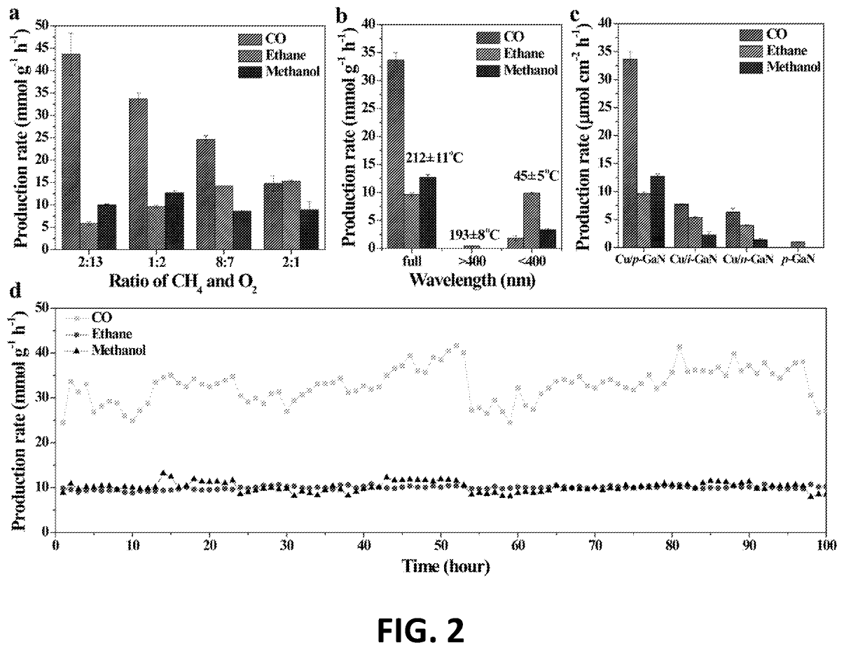

The above-described arrangements were used in a photocatalytic methane reforming reaction. A water-free flowing-bed reaction system was used to conduct the photocatalytic reaction. The effect of the CH₄:O₂ ratio in the feed gas was first investigated on the activity of photocatalyst. As shown in FIG. 2, part a, CO, C₂H₆ and CH₃OH were the main products in the photocatalytic methane reforming reaction on the Cu/p-GaN example. No observable CO₂ was found in the products. Especially, the production rate of CO was increased with the concentration of O₂ in the feed gas. The higher concentration of oxygen leads to an excessive oxidation of methane into CO. In contrast, when the concentration of oxygen in feed gas was reduced, the production rate of C₂H₆ was increased. In contrast to CO and C₂H₆, C₃OH exhibited a maximum production rate (12.8 mmol g⁻¹ h⁻¹) at a CH₄/O₂ ratio of 1:2, which was considered an optimized oxidation balance in methane reforming. Additionally, the turnover frequency (TOF) of CH₃OH production reached 1.67 h⁻¹. Compared to the gaseous and less active CO and C₂H₆, CH₃OH is more easily stored and further converted into other chemicals, thus attracting the wide attention of practical industrial applications. The achieved methanol-production activity of the Cu/p-GaN example was higher than in previous reports of methanol production with other devices. Hence, the CH₄/O₂ ratio of 1:2 was used in the following experiments.

To examine the performance of the arrangements in a photocatalytic methane reforming reaction, concentrated full-spectrum light (5,000 mW cm⁻²) from a 300 W Xe lamp was used. The full-spectrum light produced a thermal effect on the catalytic reaction due to the existence of infrared light. The temperature measurements showed that the surface temperature of the photocatalyst wafer reached 212±11° C. (FIG. 2, part b). Further investigation revealed that the increase in temperature mainly originated from the visible-infrared light (>400 nm), which contributed to a reaction temperature of 193±8° C. However, the ultraviolet light (<400 nm) only produced a weak thermal effect on photocatalyst wafer, which led to a low temperature (45±5° C.) on photocatalyst. Interestingly, the catalytic activity of the Cu/p-GaN arrangement under ultraviolet light only was observably lower than that under full-spectrum light, though the p-GaN could be photoexcited to produce holes and electrons only under ultraviolet light. Especially, the production rate of methanol under only ultraviolet light was decreased by four times. In contrast, when sole visible-infrared light was used to irradiate the photocatalyst wafer, only trace C₂H₆ was observed, though the photocatalyst wafer was heated to 193±8° C. It should be noted that the visible-infrared light (>400 nm) cannot photoexcite GaN to produce photogenerated electrons and holes due to its large band gap (3.4 eV). Hence, the visible-infrared light (>400 nm) only produces the thermal effect on the photocatalyst wafer. The above results indicate that the effective photocatalytic methane reforming under full-spectrum light involves a synergetic effect of ultraviolet and visible-infrared light wavelengths. The ultraviolet light is mainly used to photoexcite the photocatalyst to produce the photogenerated holes and electrons, while the visible-infrared light heats the photocatalyst to a high reaction temperature for assisting the activation or formation of chemical bonds. Hence, the methane reforming reaction, a photothermal catalytic reaction, can effectively improve the utilization efficiency of solar energy, thus contributing to the above high methanol-production activity.

Further investigation revealed that the Cu/p-GaN example in the photothermal catalytic methane reforming reaction exhibited a significant 3.5-fold higher activity for methanol production than the Cu/i-GaN and Cu/n-GaN arrangements (FIG. 2, part c). These results indicate that p-doping of the GaN support structure leads to higher performance in photocatalytic methane reforming relative to nearly intrinsic and n-doping. Moreover, in the absence of Cu nanoparticles, only C₂H₆ was produced on unloaded p-GaN. This indicates that p-doping and Cu nanoparticles are useful in connection with methanol production from photothermal methane reforming on GaN nanowires.

The Cu/p-GaN example also showed stable photocatalytic activity in a 100-hour photothermal catalytic reaction (FIG. 2, part d). Particularly, the production rate of methanol remained unchanged in the 100-hour photothermal catalytic reaction. A turnover number (TON) of 84,747 was achieved in the 100-hour stability test. Structural analysis of the Cu/p-GaN example after the stability testing demonstrated that the GaN nanowires were still well aligned on the silicon substrate, and the main crystal structure did not show any observable change. HAADF-STEM imaging was also used to evaluate the atomic-scale crystal structures of the GaN nanowires and CuNPs. The obtained results showed that the supported Cu cocatalyst still existed in the form of metallic CuNP, which was consistent with the results of XPS. This investigation also indicated that the Cu nanoparticles provided a stable electron-captured center in the photocatalytic reaction. The GaN nanowires also maintained the initial high crystallinity.

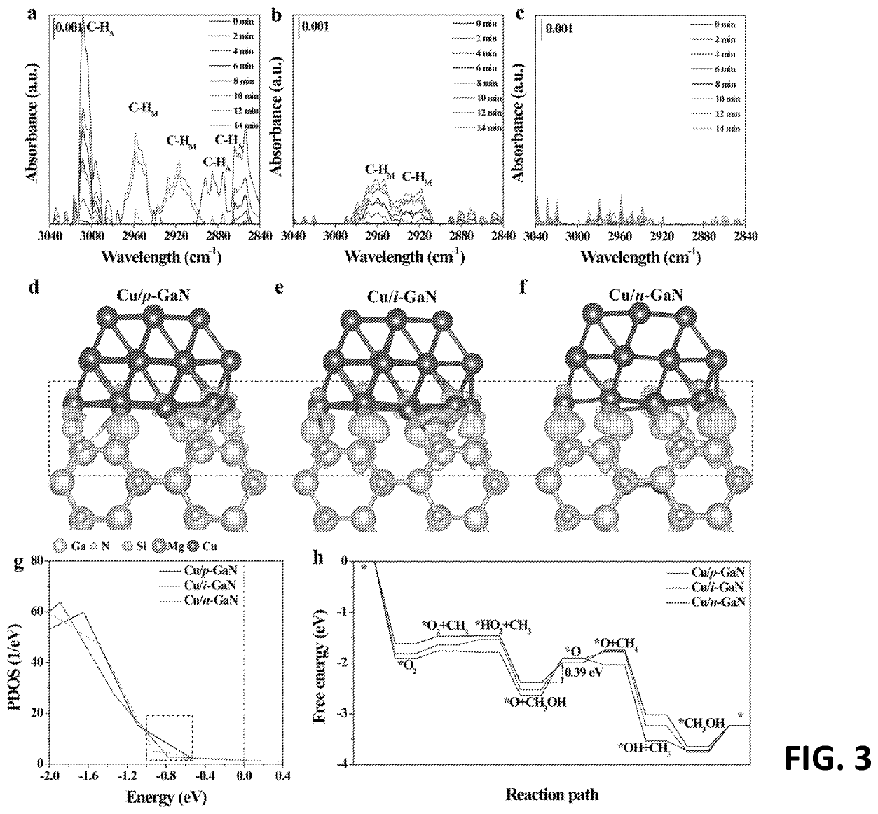

To investigate the p doping effect on the methanol-production activity of the Cu/p-GaN example, in-situ infrared diffuse reflectance infrared Fourier transform spectroscopy (DRIFTS) measurements were conducted to examine the surface catalytic methane reforming. As shown in FIG. 3, part a, in the initial stage of reaction, three relatively strong IR peaks at 3010, 2880 and 2860 cm⁻¹ appeared, which are attributed to the stretching vibration of the C—H (C—H_{A}) bond in the activated methane molecule by the photocatalyst surface. Then the intensities of those three peaks are decreased with reaction times and finally reach a balance. Meanwhile, two new IR peaks at 2960 and 2920 cm⁻¹ appeared and increased with the reaction time, probably due to the typical stretching vibration of C—H (C—H_{M}) in methanol. It should be noted that the IR peaks of C₂H₆ and CO are not observed, which is considered from the lower adsorption energy on catalyst surface and the low concentration in the flowing chamber. Compared to the Cu/p-GaN example, the Cu/i-GaN and Cu/n-GaN arrangements showed relatively weak IR peaks for the activation of methane or the production of methanol (FIG. 3, parts b and c). As such, the in-situ infrared measurements demonstrated that the p-doping in GaN significantly improves the activity of CuNP-loaded GaN by promoting methane activation and methanol formation.

The doping can significantly tune the electronic properties of the photocatalyst, which further influences the charge transfer between the photocatalyst and cocatalyst or surface catalytic reaction. Density functional theory (DFT) simulations were performed to illustrate the p doping effect on the activities of Cu/p-GaN, Cu/i-GaN and Cu/n-GaN at electronic and atomic scales. The charge density difference mappings between Cu nanoparticles and GaN surfaces showed that the charges are redistributed at the Cu-GaN interface, which revealed the metal-support interaction effect in the Cu-GaN combination (FIG. 3, parts d-f). In this metal-support interaction effect, electrons tend to be transferred to CuNP and holes to the interface region. This implies that the CuNP and GaN acted as the reducing and oxidizing regions, respectively. Especially, it is observed that almost all of electrons (skyblue regions) in the Cu/p-GaN example are aggregated on CuNP (FIG. 3, part d), indicating that p-doping significantly enhances the metal-support interaction effect between CuNP and GaN. This can greatly contribute to the charge separation and transfer in the photocatalytic reaction. However, the partial electrons in the Cu/i-GaN and Cu/n-GaN arrangements are left on the GaN layer (FIG. 3, parts e and f). Hence, p-GaN provides the supported CuNP with a higher electron density for oxygen reduction, which is also consistent with the results of XPS measurements. Moreover, the calculated projected density of states (PDOS) further demonstrates that the edge of Cu 3d states in the Cu/p-GaN example is closer to the Fermi level, allowing the Cu nanoparticles to accept electrons from GaN more easily (FIG. 3, part g).

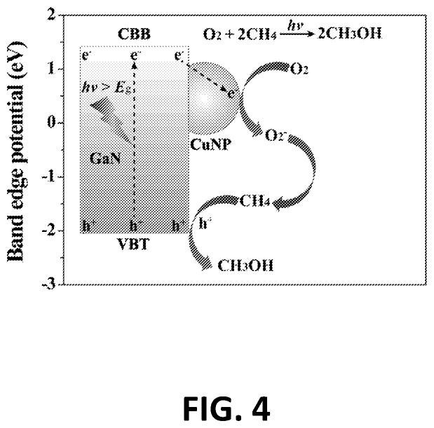

FIG. 4 shows a mechanism of photocatalytic methane reforming into methanol on Cu-loaded GaN. During photocatalytic reaction, GaN was photoexcited to produce photogenerated electrons and holes. The photogenerated electrons are then transferred to CuNP and reduce the adsorbed oxygen into the active oxygen species. Finally, the active oxygen species and the photogenerated holes synergistically oxidize and dissociate methane into the methanol.

The overall formation pathway (O₂+2CH₄→2CH₃OH) of methanol from methane reforming was also simulated to identify the rate-determining step (FIG. 3, part h). The obtained results show that the desorption step of methanol has the highest reaction energy in the pathway, which is noted as the energy barrier. Moreover, the low reaction energy in the desorption of methanol can also inhibit the undesirable oxidation of methanol and promote the regeneration of surface catalytic sites. Among those catalyst models, the Cu/p-GaN example exhibited the lowest energy barrier (0.39 eV), well explaining the highest activity of the Cu/p-GaN example for methanol production in the above testing. This result also implies that the rate-determining desorption step of methanol easily leads to the further oxidation of methanol into other carbon-based products, such as CO (O₂+CH₃OH→CO+2H₂O), observed in the above experiments. As for ethane production (O₂+4CH₄→2CH₃CH₃+2H₂O), it is attributed to the competing C—C and C—O coupling reaction. The introduction of CuNP can effectively promote the C—O coupling reaction for methanol production. The selectivity on methanol production in the photothermal methane reforming reaction was 23% though a high methanol production rate is achieved.

The above-described testing established that metal-support interaction between photocatalyst and cocatalyst was useful in photothermal catalytic methane reforming. A GaN photocatalyst having a semiconductor composition with p-doping enhanced the metal-support interaction between GaN and CuNP. The enhanced metal-support interaction promotes the electron transfer from GaN to CuNP through the CuNP-GaN interface, and also optimizes the pathway of surface catalytic reaction for methanol production.

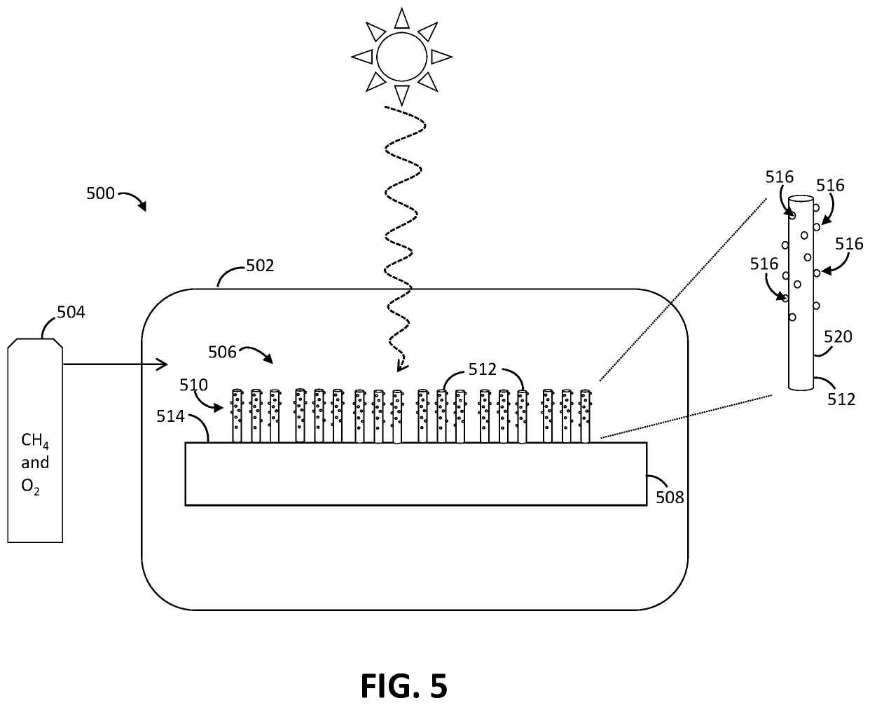

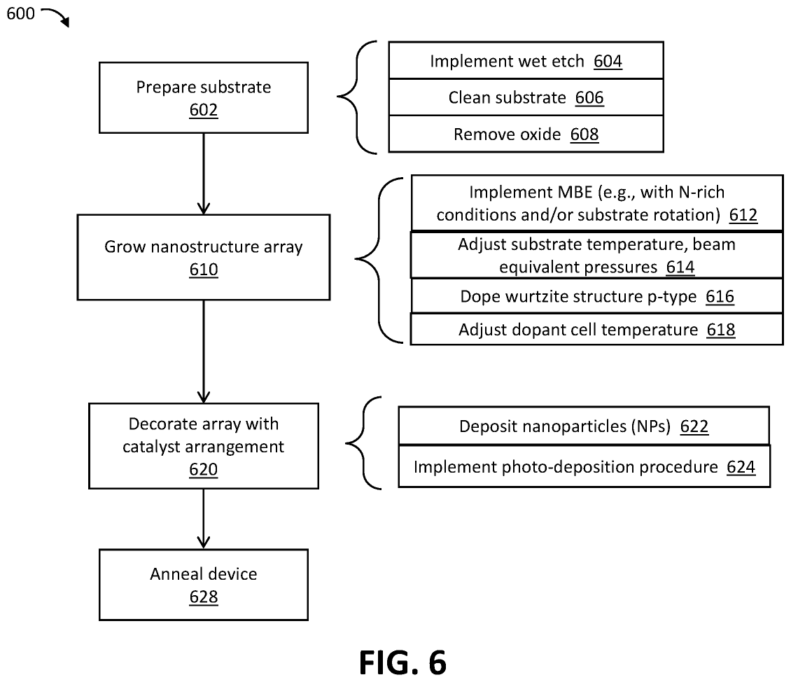

Examples of photocatalytic devices having metal-support interaction for promotion of chemical reactions, such as methane reforming into liquid fuels, are described below in connection with FIGS. 5 and 6. The metal-support interaction of the described examples may lead to a photothermal effect as described herein.

FIG. 5 depicts a photocatalytic system 500 for methane reforming in accordance with one example. Additional or alternative chemical reactions may also be implemented or supported by the system 500. In this example, the photocatalytic system 500 includes a container 502. In some cases, the container 502 is configured as a sealed reactor, such as a sealed gas-phase reactor. The container 502 may be configured to allow illumination (e.g., solar illumination) of the interior of the container 502. For instance, the container 502 may have a transparent cover, side, cap, or other portion, such as a quartz top. The size, construction, composition, configuration, and other characteristics of the container 502 may vary. The system 100 may not include a container in other cases.

In this example, solar radiation is incident upon the system 500. The manner in which the system 500 is illuminated may vary. Additional or alternative light sources may be used, including, for instance, artificial light sources.

In the example shown, the components of the system 500 are configured to implement the methane reforming in the gas phase. In other cases, the chemical reactions may operate either partially or entirely in the liquid phase.

The system 500 may include a source 504 of methane and oxygen coupled to the container 502. The manner in which the methane and oxygen are provided by the source 504 may be integrated to any desired extent.

As described herein, the system 500 does not include a voltage or other source of electrical energy. Thus, in the example of FIG. 5, the system 500 accordingly implements the methane reforming (and/or other chemical reaction) without the application of a bias voltage to a photocatalytic device of the system 500. In other cases, one or more bias voltages may be applied to one or more electrodes or other components in the system 500.

The system 500 may also be free of sacrificial agents. In the example of FIG. 5, the container 502 is sacrificial agent-free. In other cases, one or more sacrificial agents may be used to promote the methane reforming and/or other chemical reaction in the system 100.

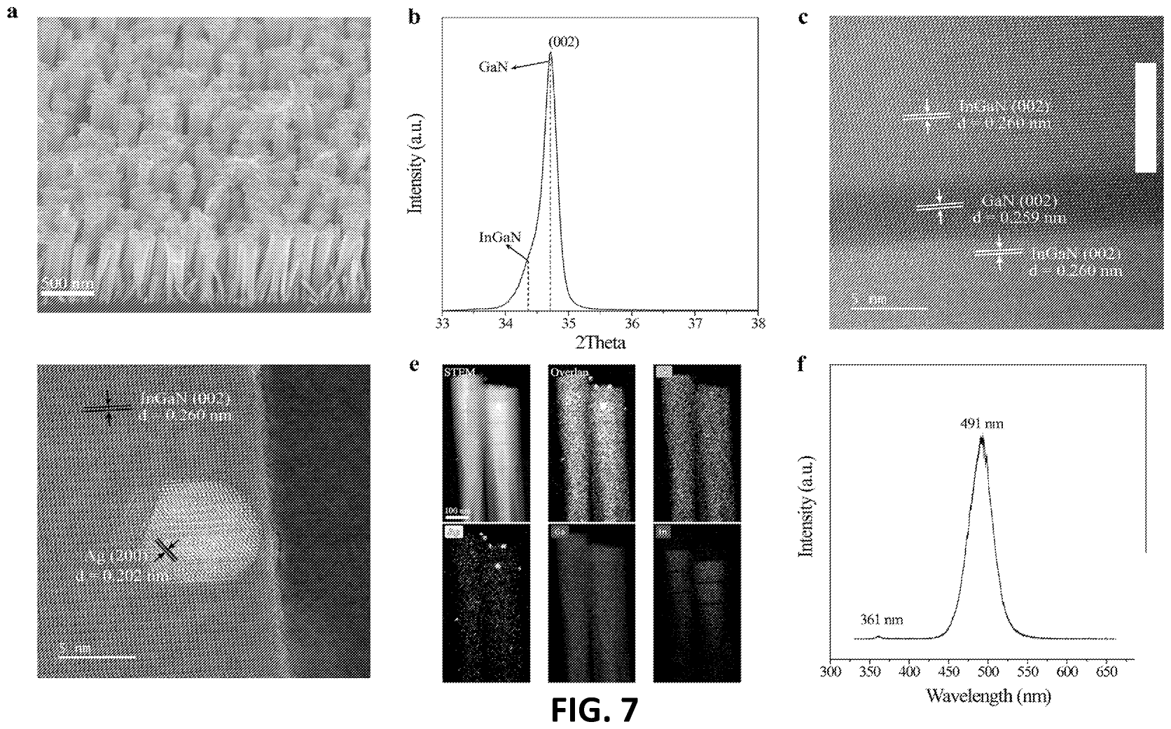

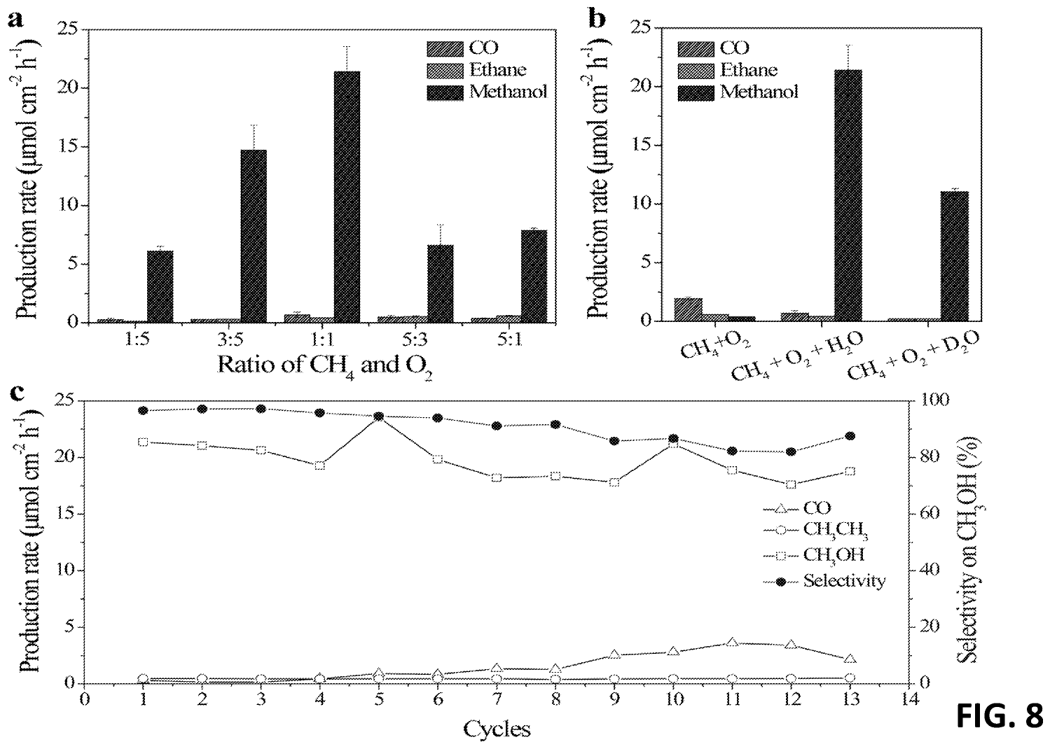

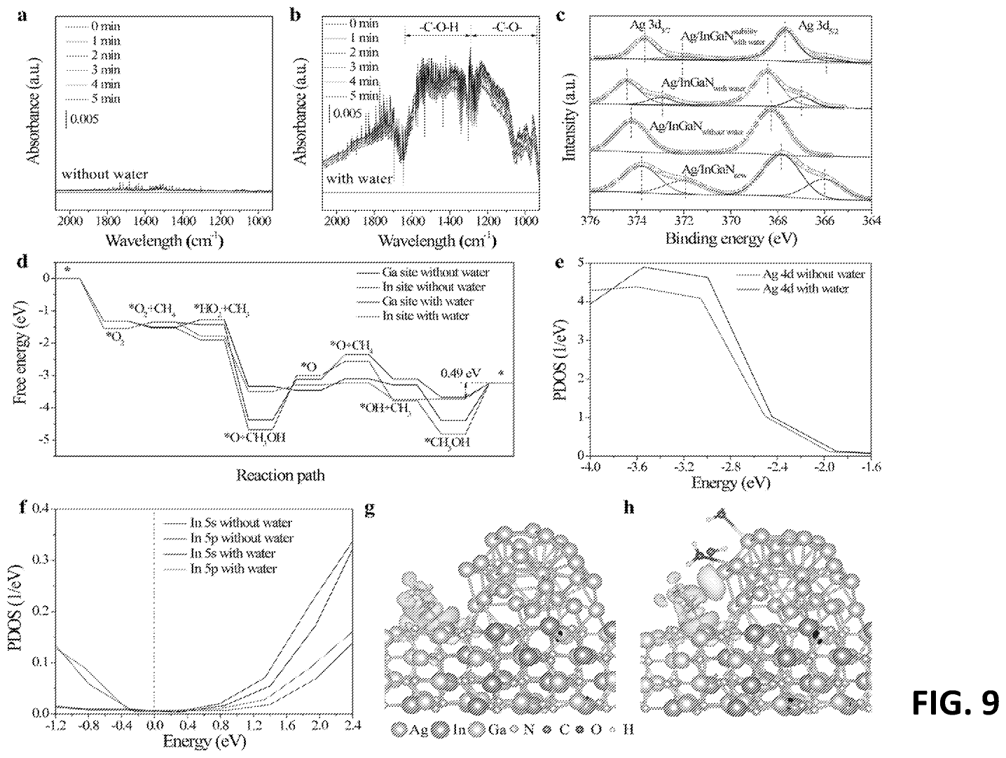

The photocatalytic system 500 includes a photocatalytic device 506 disposed in the container 502. In this case, the photocatalytic device 506 is not immersed (e.g., partially or completely) in water or other liquid. However, the photocatalytic device 506 may be configured for operation in water vapor. Examples involving water-promoted photocatalysis are described below in connection with FIGS. 7-9. In the example of FIG. 5, the photocatalytic device 506 is disposed in the container 502 in a manner to allow the incident light to illuminate the semiconductor device 506. In some cases, the photocatalytic device 506 is configured for methane reforming in response to the illumination as described herein.

The semiconductor device 506 includes a substrate 508 and an array 510 of conductive projections 512 supported by the substrate 508. In some cases, each conductive projection 512 is or includes a nanowire or other nanostructure. In this example, each conductive structure 512 is or includes a cylindrically shaped nanostructure. The cylindrical shape has a circular cross-sectional shape (e.g., a circular cylinder), as opposed to, for instance, a plate-shaped or sheet-shaped nanostructure. The conductive projections 512 may thus be configured, and/or referred to herein, as nanowires. In this example, the nanowires 512 extend outward from a top or upper surface 514 of the substrate 508. Alternative or additional surfaces of the substrate 508 may support the array 510.

The substrate 508 may be active (e.g., functional) and/or passive (e.g., structural). In one example of the former case, the substrate 508 may be or include a reflective material or layer to direct light back toward the nanowires 512. In one example of the latter case, the substrate 508 may be configured and act solely as a support structure for the nanowires 512. Alternatively or additionally, the substrate 508 may be composed of, or otherwise include, a material suitable for the growth or other deposition of the nanowires 512.

In some cases, the substrate 508 may include a light absorbing material. In such cases, the light absorbing material is configured to generate charge carriers upon solar or other illumination. The light absorbing material has a bandgap such that incident light generates charge carriers (electron-hole pairs) within the substrate. Some or all of the substrate 108 may be configured for photogeneration of electron-hole pairs.

The substrate 508 may include a semiconductor material. In some cases, the substrate 508 is composed of, or otherwise includes, silicon. For instance, the substrate 508 may be provided as a silicon wafer. The silicon may or may not be doped. The doping arrangement may vary. For example, one or more components of the substrate 508 may be non-doped (intrinsic), or effectively non-doped. The substrate 508 may include alternative or additional layers, including, for instance, support or other structural layers. The composition of the substrate 508 may thus vary. For example, the substrate may be composed of, or otherwise include, metal films, GaAs, GaN, or SiOx in other cases.

The substrate 508 may establish a surface, e.g., the surface 514, at which a catalyst arrangement (e.g., a photocatalyst arrangement) of the semiconductor device 506 is provided. The photocatalyst arrangement is provided and supported by the nanowires 512 of the array 510. In some cases, the catalyst arrangement may be a co-catalyst arrangement including a nanowire-nanoparticle architecture, as described herein.

Each nanowire 512 has a semiconductor composition for charge carrier generation in response to the incident light (e.g., solar radiation). As described herein, the semiconductor composition may include one or more semiconductor materials, e.g., III-nitride semiconductor materials, such as gallium nitride (GaN). Additional or alternative III-nitride materials may be used, including, for instance, one or more alloys of indium gallium nitride (InGaN). In some cases, each nanowire 512 includes a stack of GaN/InGaN segments. Still other III-nitride semiconductor materials may be used, including, for instance, indium nitride, indium gallium nitride, aluminum nitride, and boron nitride. Still further or alternative semiconductor materials may be used, including, for instance, aluminum oxide, gallium phosphide, gallium arsenide, indium phosphide, and silicon.

Each nanowire 512 may be or include a columnar, rod-shaped, post-shaped, or other elongated structure. The nanowires 512 may be grown or formed as described in U.S. Pat. No. 8,563,395 (“Method of growing uniform semiconductor nanowires without foreign metal catalyst and devices thereof”), the entire disclosure of which is hereby incorporated by reference. The dimensions (e.g., length, diameter), size, shape, and other characteristics of the nanowires 512 may vary.

The semiconductor composition of each nanowire 512 allows charge carriers to be generated to support the methane reforming or other chemical reaction.

As shown in FIG. 5, each nanowire 512 extends outward from the surface 514 of the substrate 508. In this example, the surface 514 of the substrate 508 is planar. Alternatively or additionally, the surface 514 of the substrate is nonplanar. In such cases, one or more subsets of the array 510 may be oriented at different angles. Examples of nonplanar substrates include various types of multi-faceted surfaces, such as a pyramidal textured surface. For instance, the pyramids of the surface 514 are square-based pyramids with four sides defined by the <111>crystallographic planes. Further details regarding examples of such nonplanar substrates and corresponding dopant gradients are provided in International Publication No. WO 2021/195484 (“Doping Gradient-Based Photocatalysis”), the entire disclosure of which is hereby incorporated by reference. The manner in, or degree to, which the surface 514 is multi-faceted or otherwise nonplanar may vary. For instance, the surface 514 may have any number of faces oriented at any angle. The pyramids or other shapes disposed along the surface 514 may be uniform or non-uniform.

The nanowires 512 may be configured to generate electron-hole pairs upon illumination. The nanowires 512 may be configured to generate the electron-hole pairs upon absorption of light at certain wavelengths (e.g., UV solar wavelengths). In some cases, each nanowire 512 may has a uniform composition. For instance, each nanowire may be composed entirely of a single semiconductor material (e.g., a III-nitride semiconductor material, such as GaN). The semiconductor composition establishes one or more bandgaps that establish the wavelength(s) at which charge carrier generation occurs. In some cases, a single bandgap is established for absorption of light of ultraviolet wavelengths in solar radiation. Other wavelengths (e.g., infrared and/or visible wavelengths) may then be used for heating as described herein.

In other cases, some or all of the nanowires 512 have multiple segments, with each segment being configured to absorb light over a respective range of wavelengths. For instance, each nanowire 512 may include a stacked or layered arrangement of semiconductor materials. Each layer in the arrangement may be configured for absorption of light at other wavelengths of the solar spectrum (e.g., infrared, visible, and/or ultraviolet wavelengths).

The layered arrangement of semiconductor materials is used to establish a multi-band structure, such as a quadruple band structure. Each layer or segment of the arrangement may have a different semiconductor composition to establish a different bandgap. For instance, in III-nitride examples, the layers or segments of the arrangement may have different indium and gallium compositions. Further details regarding the formation and configuration of multi-band structures, including, for instance, triple-band structures, are provided in U.S. Pat. No. 9,112,085 (“High efficiency broadband semiconductor nanowire devices”) and U.S. Pat. No. 9,240,516 (“High efficiency broadband semiconductor nanowire devices”), the entire disclosures of which are incorporated by reference.

Regardless of whether the nanowires 512 include more than one segment, the semiconductor composition of the nanowires 512 may have a bandgap such that solar radiation produces a thermal effect in the array 510. For instance, the absorption of IR and/or visible wavelengths of the solar spectrum produces a thermal effect, thereby heating the device 506 as described herein. The ultraviolet light in the solar radiation may then be relied upon for the photogeneration of charge carriers. In this way, solar radiation leads to both the photogeneration of charge carriers and a thermal effect. The wavelengths responsible for thermal heating and charge carrier generation may vary. For instance, the semiconductor composition may have a bandgap (e.g., one of multiple bandgaps) such that visible light does (or does not) contribute to the photogeneration of charge carriers.

The semiconductor composition of each nanowire 512 is configured to improve the efficiency of methane reforming or other chemical reaction in additional ways. For instance, the semiconductor composition of each nanowire 512 is p-type doped. The p-type doping may promote the reaction as described herein. The dopant concentration of the semiconductor composition may or may not be uniform. For instance, the dopant concentration may have a gradient within each nanowire 512 (e.g., in the vertical direction and/or the lateral direction).

In some examples the dopant may be or include magnesium. Further details regarding the manner in which magnesium doping promotes charge carrier separation and extraction are set forth in U.S. Pat. No. 10,576,447 (“Methods and systems relating to photochemical water splitting”), the entire disclosure of which is incorporated by reference. Additional or alternative dopant materials may be used, including, for instance, calcium and strontium, depending on the semiconductor light absorber of choice.

The semiconductor composition of the nanowires 512 may have a wurtzite crystal structure. For instance, the nanowires 512 may be composed of, or otherwise include, a wurtzite III-nitride semiconductor material, such as GaN. In some cases, the wurtzite crystal structure is configured with nitrogen-rich surfaces.

The photocatalytic device 506 further includes a catalyst arrangement supported by the array 510 of nanowires 512. As shown in FIG. 5, each nanowire is decorated with a catalyst arrangement. The catalyst arrangement may include a metal (e.g., copper) nanoparticles 516. The nanoparticles 516 are distributed or disposed over the array 510 of nanowires 512. A plurality of the catalyst nanoparticles 516 may be disposed on each nanowire 512, as schematically shown in FIG. 5. The nanoparticles 516 are distributed across or along the outer surface(s) of each nanowire 512. In the example of FIG. 5, the nanoparticles 516 are disposed along sidewalls 520 of the nanowires 512. Alternatively or additionally, the nanoparticles 516 are disposed along one or more other surfaces of the nanowires 512, such as a top or upper surface.

In some cases, the nanoparticles 516 are composed of, or otherwise include, a metal other than copper. For instance, additional or alternative metallic materials may be used, including, for instance, silver and gold, as well as alloys thereof.

The catalyst arrangement may be oxide-free. For instance, the surfaces of the nanoparticles do not include an oxide of the metal material. The metal oxide may remain absent despite, in some cases, the exposure of the catalyst arrangement to oxygen, e.g., in the feed gas.

The distribution of the catalyst nanoparticles 516 may be uniform or non-uniform. For instance, the catalyst nanoparticles 516 may thus be distributed uniformly in the sense that each nanowire 512 is decorated with the catalyst nanoparticles 516. The specific location of the catalyst nanoparticles 516 on each nanowire 512 may be differ from nanowire to nanowire. The schematic arrangement of FIG. 5 is shown for ease in illustration.

The nanowires 512 and the catalyst nanoparticles 516 are not shown to scale in the schematic depiction of FIG. 5. The shape of the nanowires 512 and the catalyst nanoparticles 516 may also vary from the example shown. Further details regarding the nanowires and catalyst arrangement, including the fabrication thereof, are provided below.

The nanowire and catalyst arrangement may be fabricated on a substrate (e.g., a silicon substrate) via nanostructure engineering. In one example, molecular beam epitaxial (MBE) growth of the nanowires is followed by photo-and/or other deposition of the catalyst nanoparticles 516. Further details regarding example fabrication procedures are provided below, e.g., in connection with FIG. 6.

The nanowires 512 may facilitate the methane reforming in one or more ways. For instance, each nanowire 512 may be configured to extract photogenerated electrons (e.g., electrons). The extraction brings the electrons to external sites along the nanowires 512 for use in the methane reforming or other chemical reaction. In such cases, the opposite side of the substrate 508 may be configured for hole extraction.

FIG. 6 depicts a method 600 of fabricating a photocatalytic device for methane reforming in accordance with one example. The method 600 may be used to manufacture any of the devices described herein or another device. The method 600 may include additional, fewer, or alternative acts. For instance, the method 600 may or may not include one or more acts directed to annealing the device (act 630).

The method 600 may begin with an act 602 in which a substrate is prepared or otherwise provided. The substrate may be or be formed from a silicon wafer. In one example, a 2-inch or 3-inch Si wafer was used, but other (e.g., larger) size wafers may be used. Other semiconductors and substrates may be used.

In some cases, the act 602 includes an act 604 in which a wet or other etch procedure is implemented to define the surface (e.g., nonplanar surface). For example, the etch procedure may be or include a crystallographic etch procedure. In silicon substrate examples, the crystallographic etch procedure may be or otherwise include a KOH etch procedure. In such cases, if the substrate has a <100>orientation, the wet etch procedure establishes that the surface includes a pyramidal textured surface with faces oriented along <111>planes, but additional or alternative facets may be present in some cases.

The act 602 may include fewer, additional, or alternative acts. For instance, in the example of FIG. 1, the act 602 includes an act 606 in which the substrate is cleaned (e.g., with acetone, IPA and hydrofluoric acid), and an act 608 in which oxide is removed (e.g., via annealing at about 787 degrees Celsius). The oxide removal may be implemented in the MBE reaction chamber immediately before growth.

The method 600 includes an act 610 in which a nanowire or other nanostructure array is grown or otherwise formed on the substrate. Each nanowire is formed on the surface of the substrate such that each nanowire extends outward from the surface of the substrate. Each nanowire has a semiconductor composition, as described herein. The nanowire growth may be achieved in an act 612 in which molecular beam epitaxy (MBE) is implemented. The MBE procedure may be implemented under nitrogen-rich conditions to promote the formation of N-rich surfaces (which are useful for prevention of photo-corrosion and oxidation). Alternatively or additionally, the substrate may be rotated during the MBE procedure such that each nanostructure is shaped as a cylindrically shaped nanostructure. Each nanowire may thus have a circular cross-sectional shape, as opposed to a plate-shaped or sheet-shaped nanostructure.

In some cases, the MBE procedure may be modified to fabricate the arrangement of layers or segments of each nanowire directed to providing a multi-band structure. Various parameters may be adjusted to achieve the different composition levels of the layers. For instance, the substrate temperature may be adjusted in an act 614. Beam equivalent pressures may be also adjusted in the act 614.

The act 610 includes doping the nanowires p-type in an act 616. For example, Ga and Mg fluxes (and/or other fluxes) may be controlled by using thermal effusion cells. In some cases, a dopant cell temperature is adjusted in an act 618 to control the doping (e.g., Mg doping) of the nanowires.

During the act 610, nitrogen radicals may be produced from a radio-frequency nitrogen plasma source. In one example, a nitrogen flow rate of 1.0 sccm and a forward plasma power of about 350 W were used in the growth process.

In one example, Mg-doped InGaN nanowires were grown by plasma-assisted molecular beam epitaxy (MBE) under N-rich conditions. The growth parameters included a gallium (Ga) beam equivalent pressure of about 7E-8 Torr, a nitrogen flow rate of 1 sccm, and a plasma power of 350 W. The substrate temperature, beam equivalent pressure (BEP), and magnesium (Mg) cell temperature were tuned to synthesize different single-band or multi-band InGaN nanowires with various p-doping and alloy concentrations. For instance, for single-band p-GaN nanowires or a GaN layer of a multi-band structure, the substrate temperature was 685° C., and Ga BEP was about 7E-8 Torr. The p-type doping level was tuned by using different Mg cell temperatures. For a p-In_{0.20}Ga_{0.80}N nanowire layer, the substrate temperature was 675° C., the Ga BEP was about 7E-8 Torr, and the In BEP was about 7.3E-8. For p-In_{0.27}Ga_{0.73}N nanowire layers, the substrate temperature was 662° C., the Ga BEP was about 7E-8 Torr, and the In BEP was about 7.3E-8. For p-In_{0.35}Ga_{0.65}N nanowire layers, the substrate temperature was 640° C., the Ga BEP was about 7E-8 Torr, and the In BEP was about 3.5E-8. For quadruple-band InGaN nanowires, the growth conditions are similar to those of the constituting single-band nanowires but with varying thicknesses for each segment. The substrate temperature may refer to a thermocouple reading of a substrate heater, which may be different from the actual substrate surface temperature, which may depend on the sample size, substrate holder, and mounting configuration.

The act 610 may include additional, fewer, or alternative acts. For instance, the act 610 may include one or more acts directed to forming a seed other initial layer in preparation for growth of the nanowires. The seed layer may be configured to promote the nucleation of the nanowires. In some cases, the seed layer is composed of, or otherwise includes, Ga. Further details regarding the use of seed layers are set forth below in connection with a number of examples as well as in the above-referenced patent documents.

As shown in FIG. 6, the method 600 further includes an act 620 in which the array is decorated with a co-catalyst arrangement. Dual catalysts are deposited across the array of nanowire. As described herein, the dual catalysts may be disposed in a core-shell configuration. In some cases, one of the dual catalysts is distributed as nanoparticles, while the other one is configured as a shell around or about the nanoparticles.

In the example of FIG. 6, the act 620 include an act 622 in which the nanowires are decorated with a catalyst arrangement. The act 622 may include depositing metal nanoparticles on the nanowires. The nanoparticles may be composed of, or otherwise include, copper and/or other metals, as described herein. In some cases, the deposition of the nanoparticles includes implementation of a photo-deposition procedure in an act 624. Alternative or additional deposition procedures may be used to deposit the nanoparticles, including, for instance, an e-beam evaporation procedure. Still further or alternative procedures may be used, including, for instance, other physical vapor deposition procedures, such as sputtering, as well as atomic layer deposition procedures. Further details regarding examples of the photo-deposition procedures are set forth in one or more of the above-referenced U.S. patents.

In one example, Cu nanoparticles were loaded on GaN nanowires via photo-deposition. In this case, a 0.8 cm×0.8 cm photocatalyst wafer stabilized on a Teflon holder was put on the bottom of a 390 mL chamber containing 50 mL of 20 vol % methanol aqueous solution. Then 10 μL of 0.2 mol L⁻¹ CuCl₂ (Sigma-Aldrich), i.e., the precursor of Cu nanoparticles, was added into the chamber. Before photoreduction, the chamber was covered by a vacuum-tight quartz lid and a vacuum-tight plastic ring and further evacuated. A 300 W Xe lamp (Cermax, PE300BUV) was used as the light source, which irradiated the chamber for 20 mins.

The method 600 may include one or more additional acts directed to forming the photocatalytic structures of the device. For instance, in some cases, the method 600 includes an act 628 in which the photocatalytic structures of the device are annealed. The parameters of the anneal process may vary. Alternatively or additionally, the device may be washed (e.g., in deionized water) and dried (e.g., at 150 degrees Celsius) in argon atmosphere before use (e.g., in photocatalytic methane reforming).

The order of the above-described acts of the method 600 may differ from the example shown. For instance, the annealing of the act 628 may be implemented before or after the deposition of the nanoparticles in the act 620.

Described above are renewable photocatalytic devices for methane reforming into high-value liquid fuels, such as methanol. The disclosed devices avoid challenges such as poor charge separation, sluggish methane activation and excessive oxidation that would otherwise collectively inhibit the production of methanol from photocatalytic methane reforming. The disclosed devices provide an enhanced metal-support interaction between GaN nanowire photocatalyst and Cu nanoparticle (CuNP) cocatalyst via p-doping in GaN. The CuNP-loaded p-type GaN (Cu/p-GaN) with the enhanced metal-support interaction achieved a 3.5-fold higher activity (12.8 mmol g⁻¹ h⁻¹, higher than the previous reports) for methanol production in the photothermal catalytic methane reforming with oxygen as oxidant and sunlight as the sole energy source relative to CuNP-loaded intrinsic GaN (Cu/i-GaN) or n-type GaN (Cu/n-GaN) arrangements. In-situ IR measurements indicated that the enhanced metal-support interaction significantly promotes the activation of methane and formation of methanol. Combining with X-ray photoelectron spectroscope (XPS), density functional theory (DFT) simulations demonstrated that this enhanced metal-support interaction in Cu/p-GaN greatly improves the electron transfer from p-GaN photocatalyst to the 3d states of CuNP cocatalyst through the interface between them. Catalytic pathway simulations further revealed that the enhanced metal-support interaction in Cu/p-GaN also desirably decreases the reaction energy of rate-determining methanol desorption, which decreases the excessive oxidation of produced methanol and accelerates the regeneration of surface catalytic sites. The disclosed devices thus provide a useful metal nanoparticle-loaded photocatalyst for photocatalytic methane reforming into methanol.

The devices described above may or may not be implemented in connection with water-assisted methane reforming. In contrast, described below are a number of examples of photocatalytic devices configured for water-assisted methane reforming and other chemical reactions. The disclosed devices may be used in a water-assisted photocatalytic methane oxidation process or method for highly selective methanol production. As described below, the disclosed devices may include metallic Ag nanoparticle-loaded InGaN nanowires (Ag/InGaN). Experimental XPS data and theoretical PDOS analysis demonstrated that water molecules adsorbed on the Ag nanoparticles of the disclosed devices promote electron transfer from InGaN to the Ag nanoparticles, which enabled the production of highly active Ag species with a higher reduction state in the Ag nanoparticles. Those Ag species induced by water adsorption were demonstrated to be active and useful for highly selective methanol production from the in-situ IR spectrum and reaction pathway simulation.

Examples of devices having InGaN nanowires were prepared on silicon wafers by plasma-assisted molecular beam epitaxy (PAMBE). The fabrication processes described and/or referenced above may be used. The Ag nanoparticles were deposited on InGaN nanowires by a photoreduction method, as described above. Field emission scanning electron microscopy (FESEM) images show that the InGaN nanowires with lengths of about 1.2 um are well arrayed on the silicon wafer (FIG. 1, part a). According to an X-ray diffraction (XRD) pattern (FIG. 1, part b), the crystal structure of an as-prepared sample is assigned to the hexagonal InGaN (standard PDF card 2-1078). It should be noted that one main peak at 34.7° and one weak shoulder peak at 34.4° in XRD pattern are assigned to the GaN and InGaN segments in InGaN/GaN nanowires, respectively. Additionally, the XRD peak of metallic Ag nanoparticles was not detected due to its low content. It is also revealed

that InGaN nanowires are grown along the direction on silicon wafer, which is further demonstrated by the high-angle annular dark-field scanning transmission electron microscopy (HAADF-STEM) image (FIG. 1, part c). A clear lattice fringe with a width of 0.260 nm in InGaN segment was measured along the growth direction (c-axis) of the InGaN nanowires. It should be noted that the role of GaN segment in InGaN nanowires is to stabilize the growth of InGaN segments. The metallic Ag nanoparticles with the typical lattice constant of 0.202 nm are observed on the surface of the InGaN nanowires (FIG. 1, part d), which act as a cocatalyst in the photocatalysis. The content of the Ag nanoparticles deposited on the InGaN nanowires was determined to be 45.3 μg cm⁻² according to the inductively coupled plasma-atomic emission spectrometer. The energy dispersive X-ray (EDS) elemental mapping is indicate of the distribution of the Ag nanoparticles on the surface of the InGaN nanowires as well as the formation of InGaN segments (FIG. 1, part e). The room-temperature photoluminescence spectrum shows one strong emission peak at 491 nm and one weak peak at 361 nm (FIG. 1, part f), which are assigned to the InGaN segments and the GaN segments, respectively. This result indicates that the InGaN segments have a band gap of 2.53 eV.

An example Ag/InGaN device was used in a water-assisted photocatalytic methane reforming system. In this example reaction system, the photocatalyst wafer was stabilized on a quartz holder. A water layer in the bottom of a chamber (e.g., container 502 of FIG. 5) provided water vapor in which the device is immersed for the photocatalytic reaction. CH₄ and O₂ were used as the feed gas for the methanol production.

In some cases, the ratio of CH₄ and O₂ may be optimized in the presence of water (FIG. 2, part a). The results obtained with the example Ag/InGaN device showed that the highest methanol production rate (21.4 μmol cm⁻² h⁻¹ or 45.5 μmol mg⁻¹ h⁻¹) is achieved at a ratio of 1:1. Meanwhile, a selectivity of 93.3% on methanol production was obtained. The by-products consisted of CO and ethane.

A control experiment without water demonstrated that the addition of water increases the methanol production rate by 55 times and selectivity by 9 times, shown in FIG. 2, part b. The achieved methanol-production activity on the example Ag/InGaN device was nearly one to two orders of magnitude higher than previous reports. Moreover, when replacing H₂O with D₂O, the methanol production rate is only slightly reduced (about 11.0 μmol cm⁻² h⁻¹), compared to the methane oxidation using H₂O (FIG. 2, part b), implying the crucial role of H₂O in the photocatalytic methane oxidation. It should be noted that the decreased activity with D₂O is considered to result from the lower saturated vapor pressure of D₂O relative to that of H₂O. Further nuclear magnetic resonance (NMR) measurements showed that all hydrogen elements of the produced methanol include H instead of the D from D₂O. This result implies that water molecules do not directly react with the methanol or oxygen to form the intermediate product. Instead, it is considered that the water molecule acts as a molecule “catalyst” in the photocatalytic methane reforming.

The stability of the example photocatalyst device reached 13 cycles (each cycle: 4 hours) in the presence of water (FIG. 2, part c). Stable operation of over 50 hours is significantly better than that of previously reported photocatalytic methane oxidation to methanol. A turnover number (TON) of 68,534 was also achieved in the stability testing. It should be noted that the production rate and selectivity of methanol decreased to 9.2 μmol cm⁻² h⁻¹ and 75% at the end of the stability test. However, no observable change is observed in the crystal structure of the Ag/InGaN nanowires after the stability testing. The decreased activity and selectivity on methanol is largely due to the aggregation of Ag nanoparticles on the InGaN surface according to an FESEM image.

To investigate the water promotion effect on the methanol-production activity of the Ag/InGaN device, in-situ infrared diffuse reflectance infrared Fourier transform spectroscopy (DRIFTS) measurements were performed to examine the formation of intermediates. In the absence of water, only a slight change was observed with reaction time under light irradiation (FIG. 3, part a). Besides, without irradiation, an IR peak was also not observed in the presence of water. However, with the addition of water, the observable peak appears in the range of 925-1600 cm⁻² under light irradiation, assigned to a C—O bond (FIG. 3, part b). This result indicates that the water significantly promotes the photocatalytic oxidation of methane. Moreover, the high-resolution XPS spectra of the Ag/InGaN nanowires (Ag/InGaN_{new}) before reaction shows that two kinds of Ag species exist on the Ag nanoparticles directly obtained from photoreduction (FIG. 3, part c). A highly reduced Ag species is observed at 371.9 eV. Interestingly, this peak disappears in Ag/InGaN after one cycle of methane oxidation without water addition (Ag/InGaN_{without water}). In contrast, one additional peak can still be found in the more negative region of Ag 3d XPS spectrum of the device (Ag/InGaN_{with water}) obtained from one-cycle water-assisted methane oxidation. This indicates that water stabilizes the highly reduced Ag species, though those highly reduced Ag species are unavoidably decreased with reaction time due to the aggregation of Ag nanoparticles. Especially after 16 cycles, only one weak peak was observed at 372.1 eV in Ag/InGaNstability without water). Hence, the existence of the highly reduced Ag species is considered the reason for the higher activity of the Ag/InGaN structures in water-assisted photocatalytic methane oxidation.

Density functional theory (DFT) simulations were performed to further investigate the water promotion effects on photocatalytic methane reforming into methanol. Considering the electron-aggregated effect on noble metal Ag nanoparticles during photocatalytic reaction, the Ag nanoparticles act as the cocatalyst for reducing the adsorbed oxygen into active oxygen species for activation of methane. The holes left on InGaN combine with the formed oxygen species to synergistically catalyze the methane oxidation. The free energy profile of catalytic reaction pathway shows that the desorption of methanol is the rate-determining step (FIG. 3, part d). The existence of water significantly decreases the energy barrier of methanol desorption from photocatalyst surface. The existence of water effectively renders the formation energy of C—O bond between *CH₃ and *HO₂ or *OH less negative, which avoids the excessively strong interaction between formed methanol and catalyst surface. As a result, the addition of water promotes the formation of free methanol molecules. It should be noted that the surface indium (In) site in InGaN is the more active site for methanol production compared to the surface gallium (Ga) site. Moreover, the projected density of states (PDOS) of Ag 4d states also demonstrates that water enables Ag nanoparticles with a higher reduction degree due to the distribution of Ag 4d states in a more negative region (FIG. 3, part e). Furthermore, it is revealed that the surface In site also has a lower oxidation degree in the presence of water (FIG. 3, part f), which is beneficial to inhibiting the excessive oxidation of the formed methanol into CO. This is further demonstrated by the calculated charge density difference mappings between formed methanol and photocatalyst surface. The results show that the existence of water changes the adsorption state of methanol on photocatalyst surface. In the absence of water, the formed methanol only interacts with the InGaN surface (FIG. 3, part g). However, with the addition of water, methanol is located between InGaN and the Ag nanoparticles (FIG. 3, part h). This co-interaction between methanol and the InGaN/Ag structure effectively inhibits the inert adsorption of methanol, which is beneficial to the desorption of formed methanol during catalytic reaction. Hence, the presence of water tunes the electronic states of the Ag/InGaN structure and produces more active surface sites for photocatalytic methane oxidation into methanol.

Described above are examples of devices and methods for water-promoted photocatalytic methane reforming into methanol using metallic Ag nanoparticle-loaded InGaN nanowires (Ag/InGaN), enabling methanol production from methane and oxygen with a high selectivity (>93%) and production rate (21.4 μmol cm⁻² h⁻¹ or 45.5 mmol g⁻¹ h⁻¹). Experimental XPS data and theoretical PDOS analysis revealed that water molecules adsorbed on the Ag nanoparticles (AgNP) promote electron transfer from InGaN to AgNP, which enables the formation of partial Ag species with a higher reduction state in AgNP. In-situ IR spectrum and the reaction pathway simulation data demonstrated that the newly formed Ag species induced by water adsorption are responsible for the highly selective methanol production due to the effective formation of a C-O bond and the optimal desorption of formed methanol from the surface indium site of InGaN photocatalyst. This unique water promotion effect leads to a 55-fold higher catalytic rate and 9-fold higher selectivity for methanol production compared to photocatalytic methane reforming without water addition. The disclosed devices and methods provide a useful pathway for achieving clean solar fuels from photocatalysis-based methane reforming.

The term “about” is used herein in a manner to include deviations from a specified value that would be understood by one of ordinary skill in the art to effectively be the same as the specified value due to, for instance, the absence of appreciable, detectable, or otherwise effective difference in operation, outcome, characteristic, or other aspect of the disclosed methods and devices.

The present disclosure has been described with reference to specific examples that are intended to be illustrative only and not to be limiting of the disclosure. Changes, additions and/or deletions may be made to the examples without departing from the spirit and scope of the disclosure.

The foregoing description is given for clearness of understanding only, and no unnecessary limitations should be understood therefrom.

What is claimed is:

1. A photocatalytic device comprising:
a substrate; and
an array of conductive projections supported by the substrate and extending outward from the substrate, each conductive projection of the array of conductive projections having a semiconductor composition configured for photogeneration of charge carriers;
wherein:
each conductive projection of the array of conductive projections is decorated with a catalyst arrangement;
the catalyst arrangement comprises metal nanoparticles; and
the semiconductor composition is doped p-type.

2. The photocatalytic device of claim 1, wherein the metal nanoparticles comprise copper.

3. The photocatalytic device of claim 1, wherein the semiconductor composition has a bandgap such that solar radiation produces a thermal effect in the array of conductive projections.

4. The photocatalytic device of claim 1, wherein the semiconductor composition has a bandgap such that solar radiation leads to both the photogeneration of charge carriers and a thermal effect in the array of conductive projections.

5. The photocatalytic device of claim 1, wherein the semiconductor composition has a bandgap such that visible light does not contribute to the photogeneration of charge carriers.

6. The photocatalytic device of claim 1, wherein the semiconductor composition has a bandgap such that visible light produces a thermal effect in the array of conductive projections and ultraviolet light contributes to the photogeneration of charge carriers.

7. The photocatalytic device of claim 1, wherein each conductive projection of the array of conductive projections comprises a nanowire.

8. The photocatalytic device of claim 1, wherein the semiconductor composition has a wurtzite crystal structure with nitrogen-rich surfaces.

9. The photocatalytic device of claim 1, wherein the semiconductor composition comprises GaN.

10. The photocatalytic device of claim 1, wherein the semiconductor composition comprises a III-nitride semiconductor material doped with magnesium.

11. The photocatalytic device of claim 1, wherein the semiconductor composition is uniform.

12. The photocatalytic device of claim 1, wherein the catalyst arrangement is oxide-free.

13. The photocatalytic device of claim 1, wherein the metal nanoparticles comprise silver.

14. A method of catalyzing a chemical reaction with the photocatalytic device of claim 1, the method comprising:
irradiating the photocatalytic device with radiation, the radiation comprising infrared light, visible light, and ultraviolet light; and
while the photocatalytic device is irradiated with the radiation, immersing the photocatalytic device in a feed gas.

15. The method of claim 14, wherein the feed gas comprises oxygen gas.

16. The method of claim 14, wherein the feed gas comprises methane such that the chemical reaction comprises reforming the methane into methanol.

17. The method of claim 14, wherein immersing the photocatalytic device in the feed gas comprises disposing the photocatalytic device in a flowing bed reaction system.

18. The method of claim 14, wherein irradiating the photocatalytic device comprises illuminating the photocatalytic device with solar radiation.

19. The method of claim 14, wherein irradiating the photocatalytic device is implemented such that the photocatalytic device is heated to a temperature high enough to assist in the chemical reaction.

20. The method of claim 19, wherein the temperature is above about 180 degrees Celsius.

21. The method of claim 14, wherein immersing the photocatalytic device in the feed gas comprises immersing the photocatalytic device in water vapor.

22. A method of fabricating a photocatalytic device, the method comprising:
providing a substrate having a surface;
forming an array of conductive projections on the substrate such that each conductive projection of the array of conductive projections extends outward from the substrate, each conductive projection of the array of conductive projections having a semiconductor composition configured photogeneration of charge carriers; and
decorating each conductive projection of the array of conductive projections with a catalyst arrangement,
wherein:
decorating each conductive projection of the array of conductive projections comprises depositing metal nanoparticles on each conductive projection of the array of conductive projections; and
forming the array of conductive projections comprises doping the semiconductor composition p-type.

23. The method of claim 22, wherein depositing the metal nanoparticles comprises implementing a photo-deposition procedure.

24. The method of claim 22, wherein forming the array of conductive projections comprises growing a plurality of nanowires via plasma-assisted molecular beam epitaxy (MBE) under nitrogen rich conditions.

25. A method of catalyzing a chemical reaction, the method comprising:
providing a catalytic device comprising an array of conductive projections supported by a substrate and extending outward from the substrate, each conductive projection of the array of conductive projections being decorated with a catalyst arrangement, the catalyst arrangement comprising metal nanoparticles; and
immersing the catalytic device in water vapor and a feed gas.

26. The method of claim 25, wherein the metal nanoparticles comprise silver.

27. The method of claim 25, wherein the feed gas comprises methane and oxygen such that the chemical reaction comprises reforming the methane into methanol.

28. The method of claim 25, further comprising irradiating the catalytic device while the catalytic device is immersed in the water vapor and the feed gas.

29. The method of claim 28, wherein irradiating the catalytic device comprises illuminating the catalytic device with solar radiation.
